(19) United States
(12) Patent Application Publication
(10) Pub. No.: US 2012/0282593 A1
(43) Pub. Date: Nov. 8, 2012

(54) Method and Kit for Detecting Virulent Strains of Influenza Virus

(75) Inventors: Jonathan A. McCullers, Memphis, TN (US); Julie McAuley, Thomastown (AU)

(73) Assignee: St. Jude Children's Research Hospital, Memphis, TN (US)

(21) Appl. No.: 13/098,770

(22) Filed: May 2, 2011

(51) Int. Cl. C12Q 1/70; C07H 21/04; C07K 16/08

(52) U.S. Cl. 435/5; 536/24.32; 536/24.33; 530/387.9

(57) ABSTRACT

The present invention is a kit and method for determining the virulence of an influenza virus based upon the presence or absence of leucine at position 62, arginine at position 75, arginine at position 79 and leucine at position 82 of the polymerase basic 1-F2 protein amino acid sequence.

### INTRODUCTION

This invention was made with government support under contract number R01 AI66349 awarded by the National Institutes of Health. The government has certain rights in the invention.

### BACKGROUND OF THE INVENTION

Epidemic viral infections are responsible for significant worldwide loss of life and income in human illnesses ranging from the common cold to life-threatening influenza, West Nile and HIV infections. Timely detection, diagnosis and treatment are key in limiting spread of disease in epidemic, pandemic and epizootic settings. Rapid screening and diagnostic methods are particularly useful in reducing patient suffering and population risk. Similarly, therapeutic agents that rapidly inhibit viral assembly and propagation are particularly useful in treatment regimens.

Influenza A has emerged as a potentially significant risk to human populations. Avian strains have crossed into humans and there is growing evidence that human to human spread may soon occur (Fauci (2005) Nature 435 (7041):423-424). Virology test methods for detection and confirmation of influenza A infection in a virus-secure reference laboratory, e.g., satisfying requirements for Containment Group 4 pathogens, are time consuming, high-risk and laborious, i.e., involving 4-7 days isolation of the virus in embryonated eggs; harvesting allantoic fluids from dead or dying embryos; testing the fluid in hemagglutination and hemagglutination inhibition tests, immunodiffusion; and, eventual subtyping of the virus in the fluid by hemagglutinin and neuraminidase in overnight immunodiffusion assays using specially prepared monospecific antisera. Present subtyping involves identifying each of 16 different possible viral hemagglutinin proteins in combination with 9 different possible viral neuraminidase proteins.

Rapid immunodiagnostic tests for influenza antigens include, e.g., BINAXNOW FluA and FluB (Binax, Inc., Portland, Me.), DIRECTIGEN Flu A+B (Becton Dickinson, Franklin Lakes, N.J.), FLU OIA (Biostar Inc., Boulder, Colo.), QUICKVUE (Quidel, Sand Diego, Calif.), INFLU AB QUICK (Denka Sieken Co., Ltd., Japan) and XPECT FLU A & B (Remel Inc., Lenexa, Kans.). These assays can reportedly either detect influenza A or distinguish between Influenza A and B, but importantly, not between different influenza A subtypes or between pathogenic and non-pathogenic strains of influenza A.

Recent introduction of reverse-transcriptase PCR-based diagnostics (RT-PCR) for confirming influenza A virus have resulted in important advances in diagnostics (Spackman (2005) J. Vet. Diagn. Invest. 17 (1):76-80), but because of the relative inefficiency of the reverse transcriptase enzyme and significant amounts of virus required (e.g., 10⁴ virion particles), high throughput screening of subjects with RT-PCR in an epidemic setting is not practical.

Additionally, the complexity, diversity and rapid emergence of new influenza strains has made the diagnosis of high risk strains difficult using conventional approaches. For epidemiologists, diversity resulting from high mutation rates and genetic reassortment make it challenging to anticipate where new strains may originate. Thus, there remains a significant need in the medical arts for improved, inexpensive, rapid, accurate and discriminatory methods capable of detecting strains of pathogenic viruses most often involved in generating medically important diseases.

### SUMMARY OF THE INVENTION

The present invention features methods for determining virulence of an influenza virus and diagnosing infection with a virulent strain of influenza virus by detecting the presence or absence of one or a combination of leucine at position 62, arginine at position 75, arginine at position 79 and leucine at position 82 of the amino acid sequence of polymerase basic (PB) 1-F2 protein of an influenza virus. In one embodiment, the presence or absence of one or a combination of leucine at position 62, arginine at position 75, arginine at position 79 and leucine at position 82 of the amino acid sequence of PB1-F2 protein is detected using a nucleic acid-based assay, e.g., hybridization with sequence-specific probes, a sequence-specific amplification method, direct-sequencing, denaturing gradient gel electrophoresis, single-strand conformation polymorphism analysis, or microarray analysis. In other embodiments, the presence or absence of one or a combination of leucine at position 62, arginine at position 75, arginine at position 79 and leucine at position 82 of the amino acid sequence of PB1-F2 protein is detected using a protein-based assay, e.g., an enzyme-linked solid-phase absorbent assay, a radiolabeled binding assay, a sandwich assay or an enzyme-cascade assay.

Kits containing at least one primer or probe, or at least one antibody or aptamer for detecting the presence or absence a polymerase basic (PB) 1-F2 protein or nucleic acid are also provided.

### BRIEF DESCRIPTION OF THE DRAWINGS

FIG. 1 shows an amino acid sequence comparison of PB1-F2 proteins from strains listed in Table 1. GENBANK Accession Nos. BAJ07715 (SEQ ID NO:1), BAJ07713 (SEQ ID NO:2), BAI44484 (SEQ ID NO:3), BAH24018 (SEQ ID NO:4), ACN80311 (SEQ ID NO:5), ACN76531 (SEQ ID NO:6), BAG84356 (SEQ ID NO:7), ACI89709 (SEQ ID NO:8), ABG67734 (SEQ ID NO:9), ADY80075 ((SEQ ID NO:10), ADP07194 (SEQ ID NO:11), ADU20403 (SEQ ID NO:12), ADX99442 (SEQ ID NO:13) ACU12616 (SEQ ID NO:14), ABD59819 (SEQ ID NO:15) and YP_{—}418248 (SEQ ID NO:16) are shown.

### DETAILED DESCRIPTION OF THE INVENTION

There is a need for molecular signatures to prioritize pandemic planning. It has now been found that four specific amino acid residues of polymerase basic (PB) 1-F2 protein, namely leucine 62, arginine 75, arginine 79, and leucine 82, are correlated with the virulence of influenza viruses. Specifically, these four amino acid residues enable the influenza A virus PB1-F2 protein to cause inflammation. Each amino acid contributes individually, and all are required for full virulence. Accordingly, the detection of one, two, three or all four of these amino acid residues can be used in screening for virulent influenza virus that could cause a severe pandemic. Virulent strains can then be used as vaccine candidates, targets for antivirals, etc.

PB1-F2 was identified in the course of a systematic search for influenza virus antigenic peptides presented by major histocompatibility complex class I on the surface of infected cells (Chen, et al. (2003) Virology 305:50-54). Further screening of the influenza virus genome revealed that PB1-F2 corresponded to an 87-90 amino-acid residue protein encoded by an alternate reading frame within the PB1 gene (Chen, et al. (2003) supra). The translation of PB1-F2 starts from nucleotide position 120 in the PB1 genomic segment and is believed to be initiated by ribosomal scanning (Chen, et al. (2003) supra; Lamb & Takeda (2001) Nat. Med. 7:1286-1288). PB1-F2 is maximally expressed about hours post-infection (Chen, et al. (2003) supra) and localizes to both inner and outer mitochondrial membranes, resulting in alteration of mitochondrial morphology, dissipation of mitochondrial membrane potential, and cell death. A knock out the PB1-F2 open reading frame attenuates the ability of the A/Puerto Rico/8/34 virus to induce apoptosis in immune cells (Chen, et al. (2003) supra), whereas genetically engineered virus that expresses the PB1-F2 protein from influenza virus of the 1918 pandemic on a PR8 background increases viral production rates in cells (Smith, et al. (2011) PLoS Comput. Biol. 7(2):e1001081). Further, PB1-F2 mutations have been shown to increase the pathogenicity of influenza viruses (Ozawa, et al. (2011) J. Virol. doi:10.1128/JVI.00029-11) Moreover, the basic amphipathic helix in the C-terminal region of the PB1-F2 protein has been shown to be responsible for its inner mitochondrial membrane targeting (Gibbs, et al. (2003) J. Virol. 77:7214-7224; Yamada, et al. (2004) FEBS Lett. 578:331-336) and peptides derived from the C-terminal domain were shown to have cytotoxic effects (Chanturiya, et al. (2004) J. Virol. 78:6304-6312; Zamarin, et al. (2005) PLoS Pathogens 1:e4) through BAK/BAX-mediated cytochrome release from the mitochondria (McAuley, et al. (2010) PLoS Pathol. 6(7):e1001014).

The amino acid sequence and corresponding nucleotide sequence for exemplary PB1-F2 proteins of selected influenza type A strains are listed in Table 1.

TABLE 1

| | | GENBANK Accession No. | |
|---|---|---|---|
| Strain | | Protein | Nucleotide |
| H1N1 | A/TW/3355/1997 | ABD59819 | DQ415295 |
| | A/Puerto Rico/8/34 | YP_418248 | NC_002021 |
| | A/swine/Iowa/H04YS2/2004 | ACU12616 | GQ452241 |
| H3N2 | A/Sendai-H/441/2007 | BAG84356 | AB441950 |
| | A/Taiwan/448/2007 | ACN80311 | FJ805742 |
| | A/Ohio/UR07-0126/2008 | ACN76531 | CY037893 |
| | A/swine/Minnesota/SG242/2006 | ACI89709 | CY035440 |
| | A/swine/Leipzig/1/97 | ABG67734 | DQ836173 |
| H5N1 | A/chicken/Egypt/C1Tr39/2007 | BAJ07715 | AB496998 |
| | A/duck/Egypt/D3Li748/2007 | BAJ07713 | AB496997 |
| | A/mountain hawk- | BAI44484 | AB525189 |
| | eagle/Kumamoto/1/07 | | |
| | A/Shanghai/1/2006 | BAH24018 | AB462293 |
| H1N2 | A/western grebe/Washington/ | ADP07194 | CY076163 |
| | 20569-004/2007 | | |
| | A/swine/North | ADY80075 | CY086902 |
| | Carolina/226126/2010 | | |
| H2N3 | A/mallard/Wisconsin/08OS2841/ | ADU20403 | CY079482 |
| | 2008 | | |
| | A/duck/OH/492493/2007 | ADX99442 | JF327333 |

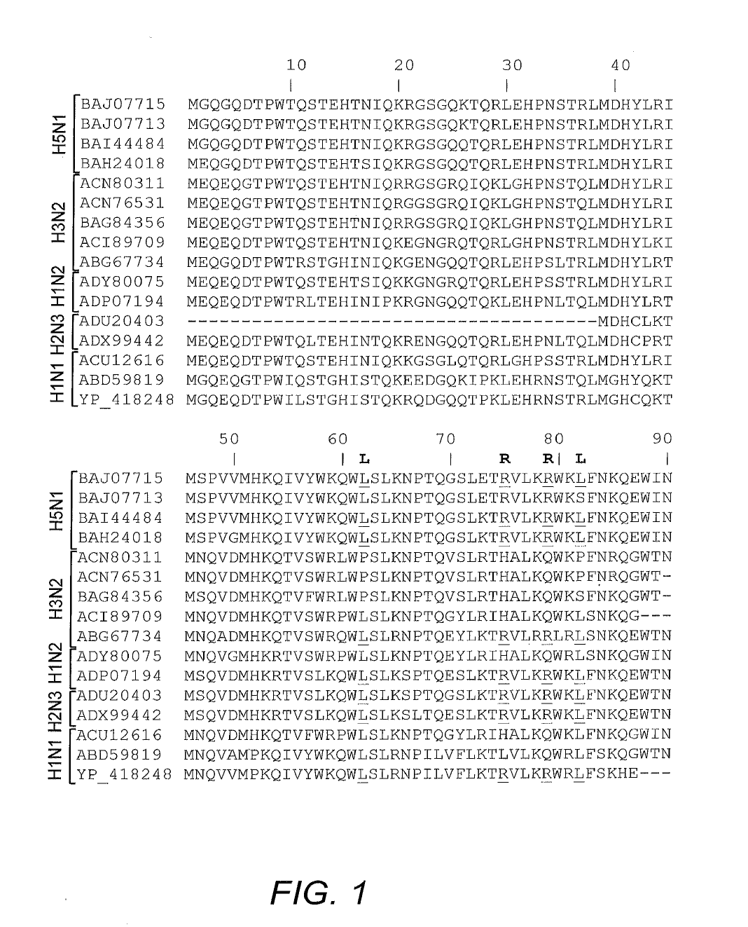

A comparison of the amino acid sequences of these proteins is shown in FIG. 1. Strains with a full-length PB1-F2 containing the combination of a leucine at position 62, a arginine at position 75, a arginine at position 79 and leucine at position 82 include A/chicken/Egypt/C1Tr39/2007, A/mountain hawk-eagle/Kumamoto/1/07, A/Shanghai/1/2006, A/western grebe/Washington/20569-004/2007, A/swine/Leipzig/1/97, A/Puerto Rico/8/34, A/mallard/Wisconsin/08052841/2008 and A/duck/OH/492493/2007. In accordance with the instant invention, these strains are considered virulent. The amino acid sequence comparison also identifies the PB1-F2 protein of strain A/duck/Egypt/D3Li748/2007 as containing the combination of a leucine at position 62, an arginine at position 75, and an arginine at position 79. While not containing all four residues (i.e., Leu62, Arg75, Arg79 and Leu82), the presence of three of the instant amino acid residues indicates that this strain could have the potential as being virulent.

Using the instant invention, virulence of strains of influenza A can be distinguished on the basis of their PB1-F2 sequence. Thus, the invention also provides methods for determining the virulence of an influenza virus by the correlation with a specific PB1-F2 sequence. Methods are also provided for determining whether a human or animal (e.g., swine, avian) subject is infected with a virulent strain of influenza virus. Assays for identifying anti-viral agents are also provided. Because the instant methods detect a protein that is produced only inside infected cells, the instant methods are useful in screening to detect subjects that are currently infected with a virulent strain of influenza virus. Advantageously, the instant methods are capable of distinguishing between the different strains of influenza A virus to identify, i.e., with a positive test result, one or more highly virulent strains of influenza A if they are present in a biological sample. Preferably, the instant test methods include steps for monitoring subjects for infection with a highly virulent strain of influenza A such as H1N1, H5N1, etc. In other embodiments, the invention provides methods for preventing the spread of a virulent influenza A virus epidemic in a plurality of human or animal subjects by identifying subjects infected with a virulent strain and treating them to prevent transmission to other subjects. Preferably, the instant methods provide for distinguishing subjects that are infected with a highly virulent influenza A strain, e.g., an H5N1, from those who are infected with a less virulent strain.

The invention additionally provides a method for determining if a subject is infected with an influenza virus, in particular whether the subject is infected with a virulent strain of influenza A virus. The method involves contacting a test sample from the subject with an agent capable of detecting one or more of the instant amino acid residues or nucleotides encoding the same and determining whether a binding interaction occurs between the instant amino acid residues or nucleotides encoding the same in the test sample and the agent. Assessing and detecting the subject binding interaction serves to determine that the test sample contains a virulent influenza virus; thereby identifying that the subject as being infected. The instant methods can also distinguish between the strains of influenza A virus, e.g., assessing whether a subject is infected with a virulent strain, or alternatively, with a lower risk avirulent strain.

Screening assays useful for identifying medicinal anti-viral compounds, e.g., in pharmaceutical development, are also provided. Thus, the invention finds uses in a variety of diagnostic and therapeutic applications.

As is conventional in the art, virulence is the degree of pathogenicity within a group or species of viruses as indicated by case fatality rates and/or the ability of the organism to invade the tissues of the host. In the context of disease, the term “virulent” is used to describe effect severity, whereas in the context of pathogens, the term “virulent” indicates the degree of infectiousness.

Influenza types A and B are typically associated with influenza outbreaks in human populations. However, type A influenza also infects other animals as well, e.g., birds and pigs. The type A viruses are categorized into subtypes based upon differences within their hemagglutinin and neuraminidase surface glycoprotein antigens. Hemagglutinin in type A viruses has 16 known subtypes and neuraminidase has 9 known subtypes. In humans, only about 3 different hemagglutinin and 2 different neuraminidase subtypes are known to establish long-term lineages, e.g., H1, H2, H3, N1, and N2. In particular, two major subtypes of influenza A have been active in humans, namely, H1N1 and H3N2; however H1N2 has emerged as a potential human pathogen.

Influenza A and influenza B viruses each contain eight segments of single stranded RNA with negative polarity. The influenza A genome encodes eleven polypeptides. Segments 1-3 encode four polypeptides, making up a RNA-dependent RNA polymerase and also the accessory protein PB1-F2. Segment 1 encodes the polymerase complex protein PB2. The remaining polymerase proteins PB1 and PA are encoded by segment 2 and segment 3, respectively. Segment 4 encodes the hemagglutinin (HA) surface glycoprotein involved in cell attachment and entry during infection. Segment 5 encodes the nucleocapsid nucleoprotein (NP) polypeptide, the major structural component associated with viral RNA. Segment 6 encodes a neuraminidase (NA) envelope glycoprotein. Segment 7 encodes two matrix proteins, designated M1 and M2, which are translated from differentially spliced mRNAs. Segment 8 encodes NS1 and NS2, two nonstructural proteins, which are translated from alternatively spliced mRNA variants. In addition, segment 2 encodes a small protein, PB1-F2, produced from an alternative reading frame within the PB1 coding region. For the purposes of the present invention, a PB1-F2 protein is a protein that shares a high degree of sequence similarity (e.g., 70%, 80%, 90% or 95% sequence similarity) and/or identity (e.g., 70%, 80%, 90% or 95% sequence identity) with one or more of the PB1-F2 proteins depicted in FIG. 1. Identity and/or similarity of two or more sequences can be determined manually or using any conventional alignment program, e.g., CLUSTALW or DIALIGN. By way of illustration, sequence comparison of the PB1-F2 proteins of strains A/TW/3355/1997 and A/Puerto Rico/8/34 indicates that these proteins share 82% sequence identity. Likewise, the PB1-F2 proteins of strains A/Moscow/10/1999, A/Panama/2007/1999, A/Sendai-H/441/2007, A/Taiwan/448/2007, and A/Ohio/UR07-0126/2008 share 90% sequence identity over their entire length and the PB1-F2 proteins of strains A/swine/Minnesota/239105/2009, A/swine/North Carolina/226126/2010, A/swine/Iowa/H04YS2/2004 and A/swine/Minnesota/SG242/2006 share 79% sequence identity over their entire length.

A PB1-F2 protein with one or more of amino acid residues Leu62, Arg75, Arg79 and Leu82 can be readily detected using a variety of techniques. In some embodiments, the instant amino acid residues are identified using a nucleic acid-based diagnostic test or assay. In other embodiments, the presence of the instant amino acid residues is detected using a protein-based diagnostic test or assay.

In accordance with the nucleic acid-based diagnostic assays, it is the codons that encode Leu62, Arg75, Arg79 and Leu82 that are detected rather than the amino acid residues themselves. For example, these diagnostic tests can use probes or primers complementary to a sequence encoding one or more of Leu62, Arg75, Arg79 and Leu82. If the nucleotides encoding one or more of Leu62, Arg75, Arg79 and Leu82 are identified as present, the influenza virus is identified as virulent. Methods of detection of nucleotide sequences of PB1-F2 can be determined in a sample by any appropriate method including, but not limited to, hybridization with sequence-specific probes or polymorphism-specific probes, sequence-specific amplification methods, direct-sequencing, denaturing gradient gel electrophoresis, single-strand conformation polymorphism analysis, and microarray analysis.

The design and use of probes for analyzing polymorphisms is described by e.g., Saiki, et al. (1986) Nature 324:163-166 and WO 89/11548. One or more probes can be designed that recognize specific sequences encoding one or more of Leu62, Arg75, Arg79 and Leu82 and hybridize to a segment of target DNA from one type of virus or viral strain but do not hybridize to the corresponding segment from another type of virus or viral strain due to the presence of different polymorphic forms in the respective segments from the two viruses. Hybridization conditions should be sufficiently stringent that there is a significant difference in hybridization intensity between sequences encoding one or more of Leu62, Arg75, Arg79 and Leu82, and preferably an essentially binary response, whereby a probe hybridizes to the nucleic acids of only those viruses that express a PB1-F2 protein with the instant amino acid residues. Some probes are designed to hybridize to a segment of target DNA such that the polymorphic site at Leu62, Arg75, Arg79 or Leu82 aligns with a central position (e.g., in a 15 mer at the 7 position; in a 16 mer, at either the 8 or 9 position) of the probe. This design of the probe achieves good discrimination in hybridization between different nucleic acids encoding PB1-F2 proteins from different viruses and/or strains.

These probes are often used in pairs, one member of a pair showing a perfect match to one reference form of a target sequence and the other member showing a perfect match to a variant form or a different reference form. Several pairs of probes can then be immobilized on the same support for simultaneous analysis of multiple polymorphisms within the same target sequence. The polymorphisms can also be identified by hybridization to nucleic acid arrays or microarrays, some examples of which are described by WO 95/11995. Examples of probes of use in detecting nucleic acids encoding Leu62, Arg75, Arg79 and Leu82 of PB1-F2 are listed in Table 2.

TABLE 2

| Sub-Type | Probe/Primer Sequence | SEQ ID NO: |
|---|---|---|
| H1N1^{a} | Leu⁶² | 17 |
| | CGATGGCTTTCCTTG | |
| | Arg⁷⁵ Arg⁷⁹ | 18 |
| | AAAACTCGTGTATTGAAACGATGGAGG | |
| | Arg⁷⁹ Leu⁸² | 19 |
| | TTGAAACGATGGAGGTTGTTCAGC | |
| H2N3^{b} | Leu⁶² | 20 |
| | CAATGGCTTTCCTTGAA | |
| | Arg⁷⁵ Arg⁷⁹ | 21 |
| | AAAACTCGTGTCTTGAAACGATGGAA | |
| | Arg⁷⁹ Leu⁸² | 22 |
| | TGAAACGATGGAAGTTGTTCAAC | |
| H5N1^{c} | Leu⁶² | 23 |
| | CAATGGCTTTCCTTGA | |
| | Arg⁷⁵ Arg⁷⁹ | 21 |
| | AAAACTCGTGTCTTGAAACGATGGAA | |
| | Arg⁷⁹ Leu⁸² | 24 |
| | TGAAACGATGGAAATTGTTCAAC | |

^{a}Sequences are from A/PR/8/34.
^{b}Sequences are from A/duck/OH/492493/2007.
^{c}Sequences are from A/Shanghai/1/2006.

With respect to a sequence-specific amplification approach, a sequence-specific primer hybridizes to a site on target DNA overlapping a polymorphism and only primes amplification of an allelic form to which the primer exhibits perfect complementarily. See Gibbs (1989) Nucleic Acid Res. 17:2427-2448. Examples of such primers are listed in Table 2. This sequence-specific primer is used in conjunction with a second primer that hybridizes at a distal site. Amplification proceeds from the two primers leading to a detectable product signifying that the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO 93/22456.

Direct sequence analysis of the nucleotides encoding PB1-F2 can be accomplished using either the dideoxy-chain termination method or the Maxam-Gilbert method (see Sambrook, et al. (2001) Molecular Cloning: A Laboratory Manual (3^{rd} Ed., CSHP, New York); Zyskind, et al. (1988) Recombinant DNA Laboratory Manual (Acad. Press).

Amplification products generated using the polymerase chain reaction can also be analyzed by the use of denaturing gradient gel electrophoresis. Different alleles of PB1-F2 can be identified based on the different sequence-dependent melting properties and electrophoretic migration of DNA in solution. Erlich, ed. (1992) PCR Technology, Principles and Applications for DNA Amplification (W.H. Freeman and Co., New York), Chapter 7.

Alleles of target sequences can also be differentiated using single-strand conformation polymorphism analysis, which identifies base differences by alteration in electrophoretic migration of single stranded PCR products, as described in Orita, et al. (1989) Proc. Nat. Acad. Sci. 86:2766-2770. Amplified PCR products can be generated as described above, and heated or otherwise denatured, to form single-stranded amplification products. Single-stranded nucleic acids may refold or form secondary structures that are partially dependent on the base sequence. The different electrophoretic mobilities of single-stranded amplification products can be related to base-sequence difference between alleles of target sequences.

The primers and/or probes of the invention may be utilized as reagents (e.g., in pre-packaged kits) for prognosis and diagnosis of influenza A infection and subtypes thereof, and in particular virulent influenza A infection.

Protein-based diagnostic tests or assays of the invention are typically carried out with one or more antibodies, aptamers or combinations thereof. The PB1-F2 proteins of the invention containing Leu62, Arg75, Arg79 and Leu82 are useful for generating antibodies for use in diagnostics and therapeutics. The antibodies can be polyclonal antibodies, distinct monoclonal antibodies or pooled monoclonal antibodies with different epitopic specificities. Monoclonal antibodies are made from antigen-containing fragments of the protein by standard procedures according to the type of antibody (see, e.g., Kohler, et al. (1975) Nature 256:495; Harlow & Lane (1988) Antibodies, A Laboratory Manual (CSHP, NY); Queen, et al. (1989) Proc. Natl. Acad. Sci. USA 86:10029-10033; WO 90/07861; WO 91/17271 and WO 92/01047. Phage display technology can also be used to mutagenize CDR regions of antibodies previously shown to have affinity for PB1-F2 proteins of the invention. Those antibodies that bind to specific PB1-F2 motifs containing one or more of Leu62, Arg75, Arg79 and Leu82 can be classified as PB1-F2-specific antibodies. Desirably, antibodies of the invention specifically bind to PB1-F2 motifs containing one or more of Leu62, Arg75, Arg79 and Leu82 without binding to other PB1-F2 proteins, i.e., those lacking Leu62, Arg75, Arg79 and/or Leu82. The antibodies can be purified, for example, by binding to and elution from a support to which the PB1-F2 protein or C-terminal peptide of PB1-F2 to which the antibodies were raised is bound.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann (1990) Clin. Exp. Immunol. 79:315-321; Kostelny, et al. (1992) J. Immunol. 148:1547-1553.

In some embodiments, the instant antibodies are pan-reactive antibodies. Pan-reactive or pan-specific antibodies are monoclonal or polyclonal antibodies that bind to any and all influenza A virus PB1-F2 proteins containing Leu62, Arg75, Arg79 and Leu82 or alternatively, that bind to more than 3 of said influenza PB1-F2 proteins, or more preferably more than 5. Desirably, the pan-reactive or pan-specific antibodies recognize PB1-F2 proteins from at least the following three influenza A strains: H1N2, H3N2, and H1N1. Pan-reactive antibodies can be used to identify the presence of an influenza A virus without identifying what subtype it is. Thus, pan-reactive monoclonal antibodies can specifically recognize conserved regions of the instant PB1-F2 proteins.

In other embodiments, the invention includes pan-reactive antibodies that are polyclonal antibodies and/or mixtures of monoclonal antibodies that, as a whole, identify all or many influenza A virus PB1-F2 proteins containing Leu62, Arg75, Arg79 and Leu82. These antibodies can recognize conserved or non-conserved regions of the PB1-F2 protein. Desirably, the mixture of antibodies preferably recognize PB1-F2 proteins that also contain regions including, but not limited to, residues QWLSL (SEQ ID NO:25), ETRVL (SEQ ID NO:26), KTRVL (SEQ ID NO:27), LKRWK (SEQ ID NO:28), LKRWR (SEQ ID NO:29), LRRLR (SEQ ID NO:30), LRLSN (SEQ ID NO:31), WRLSN (SEQ ID NO:32), WKLFN (SEQ ID NO:33) and WRLFS (SEQ ID NO:34).

Aptamers are also of use in the diagnostic methods of the invention. Aptamers are RNA or DNA molecules selected in vitro from vast populations of random sequence that recognize specific ligands by forming binding pockets. Allosteric ribozymes are RNA enzymes whose activity is modulated by the binding of an effector molecule to an aptamer domain, which is located apart from the active site. These RNAs act as precision molecular switches that are controlled by the presence or absence of a specific effector. Aptamers can bind to nucleic acids, proteins, and even entire organisms. Aptamers are different from antibodies, yet they mimic properties of antibodies in a variety of diagnostic formats. Thus, aptamers can be used instead of or in combination with antibodies to identify the presence of general and specific PB1-F2 regions.

The antibodies and/or aptamers of the invention may be utilized as reagents (e.g., in pre-packaged kits) for prognosis and diagnosis of influenza A infection and, in particular virulent influenza A infection, wherein the assay identifies the presence of PB1-F2 containing Leu62, Arg75, Arg79 and/or Leu82. If said residues are present, the influenza strain is identified as pathogenic or virulent. If said residues are not present, the influenza strain is identified as not pathogenic, less virulent or avirulent.

Representative protein-based assay formats useful for detecting influenza viruses include enzyme-linked solid-phase absorbent assays, radiolabeled binding assays, as well as, sandwich- and enzyme-cascade assay formats. Illustrative methods, as may be adaptable from the immunoassay art for use in the subject assays include homogeneous and heterogeneous assay formats; competitive and non-competitive assay formats; enzyme-linked solid phase assay formats, fluorescence assay formats, time resolved fluorescence assay formats, bioluminescent assay formats, cascade enzyme assays and the like.

In some embodiments, protein-based assay methods of the invention involve the steps of (i) separating (i.e., isolating) native viral PB1-F2 protein analyte from within a complex biological sample using a first binding agent, i.e., a capture agent; and, (ii) detecting the isolated PB1-F2 analyte using a second binding agent, i.e., a detect agent. The separating and detecting steps may be achieved using binding partners that have different levels of specificity for the PB1-F2 analyte, e.g., if the capture agent is highly specific then lesser specificity may be required in the detect reagent and vice versa. In certain embodiments, the capture agent is an anti-PB1-F2 antibody or mixture of antibodies. In certain embodiments, the PB1-F2 capture agent is bound, directly or via a linker, to a solid phase. For example, in one non-limiting example the PB1-F2 capture agent is bound to a magnetic bead. In the latter example, when brought into contact with a biological sample the PB1-F2 capture agent immobilized on the magnetic bead is effective in forming a complex with an influenza viral PB1-F2 protein in a sample. Next, a magnetic field is applied and the interaction complex, with the bound influenza virus PB1-F2 protein, is isolated from the sample. In another non-limiting example, a PB1-F2 protein capture agent is immobilized on the surface of a microtiter plate; a biological sample containing an influenza PB1-F2 protein is brought into contact with the immobilized capture reagent resulting in binding of the PB1-F2 protein to the surface of the plate; the plate is washed with buffer removing non-PB1-F2 protein viral analytes from the plate; and, the immobilized PB1-F2 protein is, thus, isolated from the biological sample. Different separation/isolation means are known, e.g., applying a magnetic field, washing and the like. The particular means employed is dependent upon the particular assay format. For example, separation may be accomplished by a number of different methods including but not limited to washing; magnetic means; centrifugation; filtration; chromatography including molecular sieve, ion exchange and affinity; separation in an electrical field; capillary action as e.g. in lateral flow test strips; immunoprecipitation; and, the like as are well-known in the art.

In certain embodiments, influenza PB1-F2 protein is separated from other viral and cellular proteins in a biological sample by bringing an aliquot of the biological sample into contact with one end of a test strip, and then allowing the proteins to migrate on the test strip, e.g., by capillary action such as lateral flow. The instant methods are distinguished from prior immunoassay methods by the presence in the assay of one or more antibodies and/or aptamers, e.g., as capture and/or detect reagents, conferring upon the assay the ability to specifically identify the presence or amount of a virulent influenza A strain of virus. Methods and devices for lateral flow separation, detection, and quantification are known in the art, e.g., U.S. Pat. Nos. 6,942,981, 5,569,608; 6,297,020; and 6,403,383. In one non-limiting example, a test strip includes a proximal region for loading the sample (the sample-loading region) and a distal test region containing a PB1-F2 protein capture agent and buffer reagents and additives suitable for establishing binding interactions between the capture agent and PB1-F2 protein in the migrating biological sample.

In alternative embodiments, a PB1-F2 protein binding agent (e.g., an antibody) conjugated with an SGC (signal generating compound) is used to detect the presence of a virulence-associated PB1-F2 protein analyte in a sample in a homogeneous assay format, i.e., without need for a separation step. In this assay method the binding of a PB1-F2 binding agent to the PB1-F2 protein induces a change in the signal produced by the SGC, e.g., a change in fluorescent anisotropy.

While a variety of competitive and non-competitive assay formats are identifiable for possible use in the instant methods, a sandwich assay format can also be used because these assays have proven performance characteristics and a variety of well-established signal amplification strategies. In such assays, a specific high affinity antibody is employed to capture a natural viral PB1-F2 antigen from within a biological sample; an anti-PB1-F2 mouse monoclonal antibody is used to detect the bound PB1-F2 antigen; and, the presence of the bound anti-PB1-F2 antibody is detected using a signal generating compound, e.g. with either an enzyme-conjugated second antibody (e.g., horse radish peroxidase-conjugated antibody; HRP) or a biotinylated second antibody and streptavidin-enzyme conjugate (e.g., HRP).

Embodiments of the invention also provide methods for distinguishing between the different strains of an Influenza A virus in a test sample based on the constituent binding properties of the PB1-F2, in which the different strains and/or subtypes of influenza A produce a distinctive pattern of binding on an array. The methods involve the steps of: (a) bringing into contact aliquots of a test sample at different predefined positions in the array; (b) detecting the presence or absence of binding at a particular position in the array; (c) determining from the pattern of binding in the array that (i) influenza PB1-F2 are present in test sample and (ii) that the pattern of PB1-F2 binding in the array constitutes a distinguishing signature for a particular strain of influenza A virus. Representative examples of the influenza A viruses that are distinguishable based in arrays include, e.g. H1N1, H2N2, H2N3, H2N5, H3N2, H3N8, H4N6, H5N1, H6N1, H6N2, H7N2, H7N3 and H7N7. By way of illustration, binding to antibodies that recognize QWLSL (SEQ ID NO:25), KTRVL (SEQ ID NO:27), LKRWR (SEQ ID NO:29) and WRLFS (SEQ ID NO:34) can indicate that the strain is an H1N1 strain. Accordingly, the antibody and/or aptamer arrays specifically identify the presence of at least one region of a virulence-associated PB1-F2, including QWLSL (SEQ ID NO:25), ETRVL (SEQ ID NO:26), KTRVL (SEQ ID NO:27), LKRWK (SEQ ID NO:28), LKRWR (SEQ ID NO:29), LRRLR (SEQ ID NO:30), LRLSN (SEQ ID NO:31), WRLSN (SEQ ID NO:32), WKLFN (SEQ ID NO:33) and WRLFS (SEQ ID NO:34).

The present invention provides methods of detecting virulence-associated PB1-F2 proteins (i.e., PB1-F2 containing Leu62, Arg75, Arg79 and Leu82) in a sample for diagnosing viral infection in a subject. In accordance with this embodiment, a biological sample is obtained from a subject, and, the presence of a virulence-associated PB1-F2 protein in the sample is determined. The presence of a detectable amount of a virulence-associated PB1-F2 protein in a sample indicates that the individual is infected with a virulent influenza virus. Any sample can be used that contains a detectable concentration of influenza proteins, in particular PB1-F2 protein. Examples of samples that can be used are lung exudates, cell extracts (respiratory, epithelial lining nose), blood, mucous, and nasal swabs, for example.

Kits are provided for carrying out the instant methods. The instant kit includes one or more primers, probes, antibodies and/or aptamers for detecting a virulence-associated PB1-F2 protein or nucleic acid and printed instructions for conducting an assay to identify a virulent influenza A virus strain in a biological sample. The instant kit optionally contains one or more of reagents, buffers or additive compositions for carrying out the instant methods. In yet other embodiments, the instant kit can further include a means, such as a device or a system, for removing the influenza viral PB1-F2 protein or nucleic acid from other potential interfering substances in the biological sample. The instant kit can further include, if desired, one or more of various components useful in conducting an assay, e.g., one or more assay containers; one or more control or calibration reagents; one or more solid phase surfaces on which to conduct the assay; or, one or more buffers, additives or detection reagents or antibodies; one or more printed instructions, e.g., as package inserts and/or container labels, for indicating the quantities of the respective components that are to be used in performing the assay, as well as, guidelines for assessing the results of the assay. The instant kit can contain components useful for conducting a variety of different types of assay formats, including, e.g., test strips, sandwich ELISA, western blot assays, latex agglutination and the like. The subject reference, control and calibrators within the instant kits can contain, e.g., one or more natural and non-natural influenza PB1-F2 proteins or nucleic acids, recombinant PB1-F2 proteins, synthetic PB1-F2 peptides, and/or appropriate calorimetric and enzyme standards for assessing the performance and accuracy of the instant methods.

The instructions for practicing the subject methods are commonly recorded on a suitable recording medium and included with the kit, e.g., as a package insert. For example, the instructions can be printed on a substrate such as paper or plastic. In other embodiments, the instructions can be digitally recorded on an electronic computer-readable storage medium, e.g., CD-ROM, diskette and the like. In yet other embodiments, instructions for conducting the instant methods can be obtained by a user from a remote digital source, e.g. at an internet website in the form of a downloadable document file.

Optionally, the kits can include reagents for performing a general test for influenza A as well as specific tests. For example a lateral flow test can have a lane for identifying the presence of a general influenza A virus and a lane for identifying whether that virus is a virulent influenza A. The general test can be any test that identified the presence of an influenza A virus, including the test for the presence of PB1-F2 protein or nucleic acid encoding the same. Alternatively, the presence of influenza A can be identified by the presence of antibodies in the blood of the patient. Moreover, PCR tests can be used to generally identify the presence of influenza A.

The reagents used in methods for detecting virulence-associated PB1-F2 as disclosed herein can similarly be used for identifying therapeutic agents that block an interaction between PB1-F2 and a binding partner of a host cell, and treating a patient with a virulent influenza A infection. Methods of screening for agents that bind to and inhibit PB1-F2 proteins can be performed in vitro using natural or synthetic PB1-F2 proteins. Alternatively, natural or synthetic PB1-F2 proteins can be used to identify agents capable of binding to PB1-F2 proteins. The instant screening assay involves contacting a virulence-associated PB1-F2 protein with a test compound and determining whether the test compound inhibits the activity of a virulence-associated PB1-F2 protein. Particularly useful screening assays employ cells which express a virulence-associated PB1-F2 protein. Such cells can be made recombinantly by co-transfection of the cells with polynucleotides encoding the proteins. In a particular embodiment, such cells are grown up in multi-well culture dishes and are exposed to varying concentrations of a test compound or compounds for a pre-determined period of time, which can be determined empirically. Whole cell lysates, cultured media or cell membranes are used for determining inhibitory activity of an agent for the virulence-associated PB1-F2 protein. Test compounds that significantly inhibit activity compared to control (e.g., a PB1-F2 protein lacking Leu62, Arg75, Arg79 and Leu82 are considered therapeutic candidates.

Isolated virulence-associated PB1-F2 proteins or fragments thereof, can be used for screening therapeutic compounds in any of a variety of drug screening techniques, wherein the PB1-F2 protein is membrane-bound, free in solution, affixed to a solid support, borne on a cell surface, or located intracellularly. A test compound is considered as an inhibitor of the virulence-associated PB1-F2 protein if the activity of the PB1-F2 is significantly lower than the activity measured in the absence of test compound. In this context, the term “significantly lower” means that in the presence of the test compound the PB1-F2 activity, when compared to that measured in the absence of test compound, is measurably lower, within the confidence limits of the assay method.

Random libraries of peptides or other compounds can be screened for suitability as inhibitors of the PB1-F2 protein. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in WO 95/12608, WO 93/06121, WO 94/08051, WO 95/35503 and WO 95/30642.

An alternative source of test compounds for use in screening for therapeutics or therapeutic leads is a phage display library. See, e.g., WO 91/18980; Key, et al., eds. (1996) Phage Display of Peptides and Proteins, A Laboratory Manual, Academic Press, San Diego, Calif. Phage display is a powerful technology that allows one to use phage genetics to select and amplify peptides or proteins of desired characteristics from libraries containing 10⁸-10⁹ different sequences. Libraries can be designed for selected variegation of an amino acid sequence at desired positions, allowing bias of the library toward desired characteristics. Libraries are designed so that peptides are expressed fused to proteins that are displayed on the surface of the bacteriophage. The phage displaying peptides of the desired characteristics are selected and can be regrown for expansion. Since the peptides are amplified by propagation of the phage, the DNA from the selected phage can be readily sequenced facilitating rapid analyses of the selected peptides.

Phage encoding peptide inhibitors can be selected by selecting for phage that bind specifically to PB1-F2 protein and/or the C-terminal end thereof. Libraries are generated fused to proteins such as gene II that are expressed on the surface of the phage. The libraries can be composed of peptides of various lengths, linear or constrained by the inclusion of two Cys amino acids, fused to the phage protein or can also be fused to additional proteins as a scaffold.

Inhibitors can also be identified from a variety of other types of libraries including RNA expression libraries, bacteriophage expression libraries, small molecule libraries, peptide libraries. Inhibitors can also be produced using the known sequence of the nucleic acid and/or polypeptide. The compounds also include several categories of molecules known to regulate gene expression, such as zinc finger proteins, ribozymes, siRNAs and antisense RNAs, which are designed using conventional methods based upon the nucleic acid sequences disclosed herein.

The above screening process can identify one or more types of inhibitors that can be incorporated into pharmaceutical compositions. These inhibitors include agents that are inhibitors of transcription, translation and post-translational processing of a virulence-associated PB1-F2 protein or inhibit or block the activity of a virulence-associated PB1-F2 protein. The agents can be prepared as conjugates in which a pharmaceutical agent or imaging component is linked to an inhibitor of a virulence-associated PB1-F2. One or more of the above entities can be combined with pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington: The Science and Practice of Pharmacy, 20th Edition. Baltimore, Md.: Lippincott Williams & Wilkins, 2000).

Pharmaceutical compositions for oral administration can be in the form of, e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. Polymeric materials can be used for oral sustained release delivery. Sustained release can be achieved by encapsulating conjugates within a capsule, or within slow-dissolving polymers. Preferred polymers include sodium carboxymethylcellulose, hydroxypropylcellulose, hydroxypropylmethylcellulose and hydroxyethylcellulose. Other preferred cellulose ethers have been described (Alderman (1984) Int. J. Pharm. Tech. & Prod. Mfr. 5(3):1-9). Factors affecting drug release have been described in the art (Samba, et al. (1979) Int. J. Pharm. 2:307). For administration by inhalation, the compounds for use according to the disclosures herein are conveniently delivered in the form of an aerosol spray preparation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Effective dosage amounts and regimes (amount and frequency of administration) of the pharmaceutical compositions are readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

A compound can be administered to a patient for prophylactic and/or therapeutic treatments. A therapeutic amount is an amount sufficient to remedy a disease state or symptom, or otherwise prevent, hinder, retard, or reverse the progression of disease or any other undesirable symptom in any way whatsoever. In prophylactic applications, a compound is administered to a patient susceptible to or otherwise at risk of a particular disease or infection. Hence, a “prophylactically effective” amount is an amount sufficient to prevent, hinder or retard a disease state or its symptoms. In either instance, the precise amount of compound contained in the composition depends on the patient's state of health and weight.

In prophylactic application, pharmaceutical compositions or medicants are administered to a patient susceptible to, or otherwise at risk for developing influenza A infection in an amount sufficient to prevent, reduce, or arrest the development of influenza A infections. In therapeutic applications, compositions or medicants are administered to a patient suspected to develop, or already suffering from influenza in an amount sufficient to reverse, arrest, or at least partially arrest, the symptoms of influenza A infections. In both prophylactic and therapeutic regimes, active agents in the form of inhibitors of virulence associated PB1-F2 are usually administered in several dosages until a sufficient response has been achieved. However, in both prophylactic and therapeutic regimes, the active agents can be administered in a single dosage until a sufficient response has been achieved. Typically, the treatment is monitored and repeated dosages can be given. Furthermore, the treatment regimes can employ similar dosages; routes of administration and frequency of administration to those used in treating influenza A infection or progression of an influenza A infection.

What is claimed is:

1. A method for determining virulence of an influenza virus comprising detecting the presence or absence of at least one of leucine at position 62, arginine at position 75, arginine at position 79 or leucine at position 82 of the amino acid sequence of polymerase basic (PB) 1-F2 protein of an influenza virus, wherein the presence of at least one of leucine at position 62, arginine at position 75, arginine at position 79 or leucine at position 82 of PB1-F2 protein indicates that the influenza virus is virulent.

2. The method of claim 1, wherein the presence or absence of at least one of leucine at position 62, arginine at position 75, arginine at position 79 or leucine at position 82 of the amino acid sequence of PB1-F2 protein is detected using a nucleic acid-based assay.

3. The method of claim 2, wherein the nucleic acid-based assay comprises hybridization with sequence-specific probes, a sequence-specific amplification method, direct-sequencing, denaturing gradient gel electrophoresis, single-strand conformation polymorphism analysis, or microarray analysis.

4. The method of claim 1, wherein the presence or absence of at least one of leucine at position 62, arginine at position 75, arginine at position 79 or leucine at position 82 of the amino acid sequence of PB1-F2 protein is detected using a protein-based assay.

5. The method of claim 4, wherein the protein-based assay comprises an enzyme-linked solid-phase absorbent assay, a radiolabeled binding assay, a sandwich assay or an enzyme-cascade assay.

6. The method of claim 1, wherein the presence or absence of a combination of any two of the amino acids selected from the group consisting of leucine at position 62, arginine at position 75, arginine at position 79 and leucine at position 82 is detected.

7. The method of claim 1, wherein the presence or absence of a combination of any three of the amino acids selected from the group consisting of leucine at position 62, arginine at position 75, arginine at position 79 and leucine at position 82 is detected.

8. The method of claim 1, wherein the presence or absence of the combination of leucine at position 62, arginine at position 75, arginine at position 79 and leucine at position 82 is detected.

9. A method for diagnosing infection with a virulent strain of influenza virus comprising detecting in a biological sample the presence or absence of at least one of leucine at position 62, arginine at position 75, arginine at position 79 or leucine at position 82 of the amino acid sequence of polymerase basic (PB) 1-F2 protein of an influenza virus, wherein the presence of at least one of leucine at position 62, arginine at position 75, arginine at position 79 or leucine at position 82 of PB1-F2 protein indicates that the influenza virus is virulent.

10. The method of claim 9, wherein the presence or absence of at least one of leucine at position 62, arginine at position 75, arginine at position 79 or leucine at position 82 of the amino acid sequence of PB1-F2 protein is detected using a nucleic acid-based assay.

11. The method of claim 10, wherein the nucleic acid-based assay comprises hybridization with sequence-specific probes, a sequence-specific amplification method, direct-sequencing, denaturing gradient gel electrophoresis, single-strand conformation polymorphism analysis, or microarray analysis.

12. The method of claim 9, wherein the presence or absence of at least one of leucine at position 62, arginine at position 75, arginine at position 79 or leucine at position 82 of the amino acid sequence of PB1-F2 protein is detected using a protein-based assay.

13. The method of claim 12, wherein the protein-based assay comprises an enzyme-linked solid-phase absorbent assay, a radiolabeled binding assay, a sandwich assay or ah enzyme-cascade assay.

14. The method of claim 9, wherein the presence or absence of the combination of leucine at position 62, arginine at position 75, arginine at position 79 and leucine at position 82 is detected.

15. A kit comprising at least one primer or probe for detecting the presence or absence a nucleic acid encoding a polymerase basic (PB) 1-F2 protein comprising at least one of leucine at position 62, arginine at position 75, arginine at position 79 or leucine at position 82 of the PB1-F2 amino acid sequence.

16. The kit of claim 15, wherein the presence or absence of the combination of leucine at position 62, arginine at position 75, arginine at position 79 and leucine at position 82 is detected.

17. A kit comprising at least one antibody or aptamer for detecting the presence or absence a polymerase basic (PB) 1-F2 protein comprising at least one of leucine at position 62, arginine at position 75, arginine at position or leucine at position 82 of the PB1-F2 amino acid sequence.

18. The kit of claim 17, wherein the presence or absence of the combination of leucine at position 62, arginine at position 75, arginine at position 79 and leucine at position 82 is detected.


SEQUENCE LISTING (34 sequences)

SEQ ID NO: 1 - protein, 90 aa, Influenza A virus
MGQGQDTPWTQSTEHTNIQKRGSGQKTQRLEHPNSTRLMDHYLRIMSPVVMHKQIVYWKQWLSLKNPTQGSLETRVLKRWKLFNKQEWIN

SEQ ID NO: 2 - protein, 90 aa, Influenza A virus
MGQGQDTPWTQSTEHTNIQKRGSGQKTQRLEHPNSTRLMDHYLRIMSPVVMHKQIVYWKQWLSLKNPTQGSLETRVLKRWKSFNKQEWIN

SEQ ID NO: 3 - protein, 90 aa, Influenza A virus
MGQGQDTPWTQSTEHTNIQKRGSGQQTQRLEHPNSTRLMDHYLRIMSPVVMHKQIVYWKQWLSLKNPTQGSLKTRVLKRWKLFNKQEWIN

SEQ ID NO: 4 - protein, 90 aa, Influenza A virus
MEQGQDTPWTQSTEHTSIQKRGSGQQTQRLEHPNSTRLMDHYLRIMSPVGMHKQIVYWKQWLSLKNPTQGSLKTRVLKRWKLFNKQEWIN

SEQ ID NO: 5 - protein, 90 aa, Influenza A virus
MEQEQGTPWTQSTEHTNIQRRGSGRQIQKLGHPNSTQLMDHYLRIMNQVDMHKQTVSWRLWPSLKNPTQVSLRTHALKQWKPFNRQGWTN

SEQ ID NO: 6 - protein, 89 aa, Influenza A virus
MEQEQGTPWTQSTEHTNIQRGGSGRQIQKLGHPNSTQLMDHYLRIMNQVDMHKQTVSWRLWPSLKNPTQVSLRTHALKQWKPFNRQGWT

SEQ ID NO: 7 - protein, 89 aa, Influenza A virus
MEQEQGTPWTQSTEHTNIQRRGSGRQIQKLGHPNSTQLMDHYLRIMSQVDMHKQTVFWRLWPSLKNPTQVSLRTHALKQWKSFNKQGWT

SEQ ID NO: 8 - protein, 87 aa, Influenza A virus
MEQEQDTPWTQSTEHTNIQKEGNGRQTQRLGHPNSTRLMDHYLKIMNQVDMHKQTVSWRPWLSLKNPTQGYLRIHALKQWKLSNKQG

SEQ ID NO: 9 - protein, 90 aa, Influenza A virus
MEQGQDTPWTRSTGHINIQKGENGQQTQRLEHPSLTRLMDHYLRTMNQADMHKQTVSWRQWLSLRNPTQEYLKTRVLRRLRLSNKQEWTN

SEQ ID NO: 10 - protein, 90 aa, Influenza A virus
MEQEQDTPWTQSTEHTSIQKKGNGRQTQRLEHPSSTRLMDHYLRIMNQVGMHKRTVSWRPWLSLKNPTQEYLRIHALKQWRLSNKQGWIN

SEQ ID NO: 11 - protein, 90 aa, Influenza A virus
MEQEQDTPWTRLTEHINIPKRGNGQQTQKLEHPNLTQLMDHYLRTMSQVDMHKRTVSLKQWLSLKSPTQESLKTRVLKRWKLFNKQEWTN

SEQ ID NO: 12 - protein, 52 aa, Influenza A virus
MDHCLKTMSQVDMHKRTVSLKQWLSLKSPTQGSLKTRVLKRWKLFNKQEWTN

SEQ ID NO: 13 - protein, 90 aa, Influenza A virus
MEQEQDTPWTQLTEHINTQKRENGQQTQRLEHPNLTQLMDHCPRTMSQVDMHKRTVSLKQWLSLKSLTQESLKTRVLKRWKLFNKQEWTN

SEQ ID NO: 14 - protein, 90 aa, Influenza A virus
MEQEQDTPWTQSTEHINIQKKGSGLQTQRLGHPSSTRLMDHYLRIMNQVDMHKQTVFWRPWLSLKNPTQGYLRIHALKQWKLFNKQGWIN

SEQ ID NO: 15 - protein, 90 aa, Influenza A virus
MGQEQGTPWIQSTGHISTQKEEDGQKIPKLEHRNSTQLMGHYQKTMNQVAMPKQIVYWKQWLSLRNPILVFLKTLVLKQWRLFSKQGWTN

SEQ ID NO: 16 - protein, 87 aa, Influenza A virus
MGQEQDTPWILSTGHISTQKRQDGQQTPKLEHRNSTRLMGHCQKTMNQVVMPKQIVYWKQWLSLRNPILVFLKTRVLKRWRLFSKHE

SEQ ID NO: 17 - DNA, 15 bp, Artificial sequence
  Synthetic oligonucleotide
cgatggctttccttg

SEQ ID NO: 18 - DNA, 27 bp, Artificial sequence
  Synthetic oligonucleotide
aaaactcgtgtattgaaacgatggagg

SEQ ID NO: 19 - DNA, 24 bp, Artificial sequence
  Synthetic oligonucleotide
ttgaaacgatggaggttgttcagc

SEQ ID NO: 20 - DNA, 17 bp, Artificial sequence
  Synthetic oligonucleotide
caatggctttccttgaa

SEQ ID NO: 21 - DNA, 26 bp, Artificial sequence
  Synthetic oligonucleotide
aaaactcgtgtcttgaaacgatggaa

SEQ ID NO: 22 - DNA, 23 bp, Artificial sequence
  Synthetic oligonucleotide
tgaaacgatggaagttgttcaac

SEQ ID NO: 23 - DNA, 16 bp, Artificial sequence
  Synthetic oligonucleotide
caatggctttccttga

SEQ ID NO: 24 - DNA, 23 bp, Artificial sequence
  Synthetic oligonucleotide
tgaaacgatggaaattgttcaac

SEQ ID NO: 25 - protein, 5 aa, Artificial sequence
  Synthetic peptide
QWLSL

SEQ ID NO: 26 - protein, 5 aa, Artificial sequence
  Synthetic peptide
ETRVL

SEQ ID NO: 27 - protein, 5 aa, Artificial sequence
  Synthetic peptide
KTRVL

SEQ ID NO: 28 - protein, 5 aa, Artificial sequence
  Synthetic peptide
LKRWK

SEQ ID NO: 29 - protein, 5 aa, Artificial sequence
  Synthetic peptide
LKRWR

SEQ ID NO: 30 - protein, 5 aa, Artificial sequence
  Synthetic peptide
LRRLR

SEQ ID NO: 31 - protein, 5 aa, Artificial sequence
  Synthetic peptide
LRLSN

SEQ ID NO: 32 - protein, 5 aa, Artificial sequence
  Synthetic peptide
WRLSN

SEQ ID NO: 33 - protein, 5 aa, Artificial sequence
  Synthetic peptide
WKLFN

SEQ ID NO: 34 - protein, 5 aa, Artificial sequence
  Synthetic peptide
WRLFS